(19) United States
(12) Patent Application Publication
(10) Pub. No.: US 2005/0004449 A1
(43) Pub. Date: Jan. 6, 2005

(54) Method for marker-less navigation in preoperative 3D images using an intraoperatively acquired 3D C-arm image

(76) Inventors: Matthias Mitschke, Nurnberg (DE); Norbert Rahn, Forchheim (DE); Dieter Ritter, Furth (DE)

Correspondence Address: SCHIFF HARDIN LLP;Patent Department, 6600 Sears Tower, 233 South Wacker Drive, Chicago, IL, 60606, US

(21) Appl. No.: 10/849,694

(22) Filed: May 20, 2004

(30) Foreign Application Priority Data
May 20, 2003 (DE) 103 22 739.3

(51) Int. Cl. A61B 5/05

(52) U.S. Cl. 600/424; 600/427

(57) ABSTRACT

In a method for marker-less navigation of a medical instrument in preoperative 3D images using an intraoperatively acquired 3D C-arm image, an intraoperative 3D Image is acquired with a C-arm system, the medical instrument brought into registration with regard to the intraoperative 3D image D, whereby a registration matrix MDN is obtained. The intraoperative 3D image is brought into registration with regard to an existing preoperative 3D image by means of image-based registration, whereby a registration matrix is obtained. Navigation of the medical instrument in the preoperative 3D image can then proceed.

### BACKGROUND OF THE INVENTION

1. Field of the Invention

The present invention concerns a method for navigation (for example of a medical instrument) in a preoperatively acquired 3D image. The invention in particular concerns navigation that does not require anatomical and/or artificial markers.

2. Description of the Prior Art

Minimally invasive (meaning with least possible operative complexity) examinations or treatments of a diseased patient ensue to an increasing degree. Examples are treatments with endoscopes, laparoscopes or catheters, which respectively are inserted into the examination region of the patient via a small opening in the body. Catheters, for example, are frequently used in cardiological examinations.

A problem from the medical-technical point of view is that, during the operation (examination), the medical instrument (in the following, a catheter will be discussed as a non-limiting example) can be visualized very exactly and with high resolution during the intervention using intraoperative x-ray supervision with a C-arm system, but the anatomy of the patient can only be very insufficiently imaged during the intervention. In the framework of operation planning, however, the doctor often desires to show the Medical Instrument in a 3D image (3D data set) acquired before the intervention (preoperatively).

German OS 101 51 438 discloses a method for intraoperative 3D imaging, in particular with the use of small, mobile C-arm systems, wherein a preoperatively generated 3D image dataset with a large volume is registered without markers using an intraoperatively generated small-volume 3D image dataset.

### SUMMARY OF THE INVENTION

An object of the present invention is to be able to visualize the real position and location of medical instruments in a simple manner in preoperatively acquired 3D images, in order to be able to navigate the instrument in the preoperatively acquired 3D image.

This object is achieved according to the invention by a method for marker-less navigation of a medical instrument in preoperative 3D images using an intraoperatively acquired 3D C-arm image, including the steps of acquiring an intraoperative 3D image with a C-arm, x-ray system bringing the medical instrument into registration with the intraoperative 3D image, whereby a registration matrix is obtained, bringing the intraoperative 3D image into registration with an existing preoperative 3D image by means of image-based registration, whereby a registration matrix is obtained, and navigating the medical instrument in the preoperative 3D image.

The preoperative 3D image is inventively acquired in a first step.

The registration matrix is obtained by marker-less registration.

Furthermore, the registration matrix is obtained by an image-based registration of the preoperative 3D image with regard to the intraoperative 3D image.

Deformation of the C-arm system is inventively taken into account in the determination of the registration matrix.

According to the present invention, results of an operation plan are taken into account in the navigation in the preoperative 3D image.

The above object also is achieved in accordance with the invention in a C-arm x-ray device operating according to the above-described method.

### DESCRIPTION OF THE DRAWINGS

FIG. 1 schematically illustrates the basic components of an inventive medical examination and/or treatment apparatus,

FIG. 2 illustrates the principle of marker-based registration of two 3D images.

FIG. 3 is flowchart of the inventive method.

### DESCRIPTION OF THE PREFERRED EMBODIMENTS

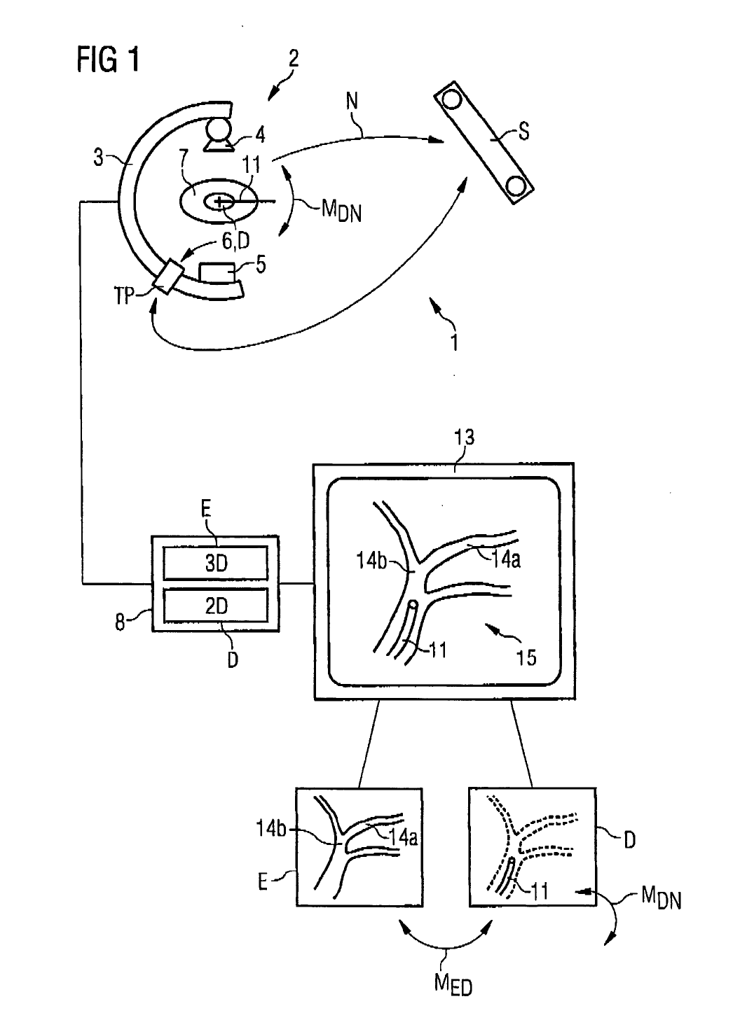

FIG. 1 shows the basic components and operating principle of an inventive examination and/or treatment apparatus 1. The apparatus has an acquisition device 2 for acquiring two-dimensional fluoroscopic images (2D fluoro-images) or for acquiring a 3D dataset (that is, however, composed of a group of 2D fluoro-images). The examination and/or treatment apparatus 1 has a C-arm 3 on which an x-ray source 4 and a radiation detector 5, for example a solid-state image detector are mounted. A tool plate TP also is mounted on the C-arm 3. The examination region 6 of a patient 7 preferably is located in the isocenter of the C-arm 3, so that it can be seen in full form in a 3D image D that is intraoperatively acquired by means of the C-arm 3.

Located in direct proximity to the acquisition device 2 is a navigation sensor S, with which the current position of the tool plate TP (and thus the position of a 3D image intraoperatively acquired by means of the C-arm 3 and the position and location of a medical instrument 11 used for the operation) can be detected.

The operation of the apparatus 1 is controlled by a control and processing device 8 that, among other things, also controls the image acquisition operation. It also has an image processing device (not shown in detail). In this, among other things, a 3D image dataset E is present that has been preoperatively acquired. This preoperative dataset F can have been acquired with an arbitrary imaging modality, for example with a computed tomography apparatus CT, a magnetic resonance tomography apparatus MRT, a ultrasound apparatus UR, a nuclear medicine apparatus NM, a positron emission tomography apparatus PET, etc. A 3D image D intraoperatively acquired with the C-arm apparatus 2, the position of which is precisely defined relative to the navigation system S, exists in the image processing device.

In the shown example, a catheter 11 (as a representative of any suitable medical instrument) is inserted into the examination region 6, here the heart. The position and location of this catheter 11 can be detected by the navigation system S and can be visualized in its current position and location in the intraoperative 3D image D. The position of the medical instrument 11 in the 3D image D can be specified by a matrix MDN. Such a 3D image is shown enlarged in FIG. 1 below, as a schematic representation.

The present invention provides a method in which the catheter 11 is mixed into the preoperative 3D image E, allowing the catheter 11 to be navigated in the 3d image E. The preoperative 3D image can have been measured by means of an acquisition method (high-resolution, functional, etc.) specific to the current clinical problem.

In the inventive method, an imaging rule is first set as to how the medical instrument 11 is imaged in the intraoperative 3D image D acquired in an arbitrary position of the C-arm 3, so it can be navigated. Such an imaging rule is—as already mentioned—specified by a matrix MDN and also designated as a “registration”. A further imaging rule (registration) is then sought as to how the intraoperative 3D image D, which includes the catheter 11, is correlated relative to the preoperative 3D image. Such an imaging rule or correlation is specified by the matrix MED. By chaining both imaging rules or matrices, a transformation N of the catheter 11 in the 3D image E is possible.

The result of such a transformation is shown in FIG. 1 in the form of an image 15 shown superimposed on a monitor 13, in which the instrument 11 as well as both 3D Images E and D have been fused or superimposed.

In order to be able to realize a correct (positionally correct) superimposition of an intraoperative 3D image with a preoperative 3D image E, it is necessary to register both images relative to one another, or respectively relative to the navigation sensor S, meaning to correlate their coordinate systems with one another. A registration is—as mentioned above—an image rule, and defines the location of a coordinate system in a coordinate system, or with regard to another coordinate system. Such a registration is also known as a “matching”. Such a registration can ensue, for example, interactively on the screen with the user.

To register two 3D images, different possibilities are possible:

1. A first possibility is, in the first of the two 3D images, to identify a reasonable number of image elements, and to identify the same image element or image elements in the second 3D image, and then to align this second 3D image by translation and/or rotation and/or 2D projection with regard to the first 3D image, such that the content structure of both 3D images can be brought into congruence, Such image elements are designated as “landmark” and can be of anatomical origin or have been artificially applied. Markers of anatomical origin—such as, for example, vessel branching points, small sections of coronary arteries, but also corners of the mouth or the tip of the nose—are designated as “anatomical markers” Artificial markers are, for example, screws that are placed in a preoperative operation, or even simple objects that are attached (for example glued) to the body surface. Anatomical or artificial markers can be interactively set by the user (for example by clicking on the screen) in the first 3D image and subsequently sought and identified in the second 3D image by suitable analysis algorithms. Such a registration is designated as a “marker-based registration”.

2. A further possibility is known as the “image-based registration”. The 3D images (each in the form of a cube) are arranged one behind the other (by computer, for example on the screen), projected onto one another by means of a parallel ray beam, and the correlation is determined. One of the two cubes is rotated and/or shifted and/or stretched until the correlation exhibits a minimal deviation. Directed by the operator, the moved cube is first brought into a position in which it is most similar to the second cube and then the optimization cycle is initiated, in order to thus shorten the calculation time for the registration.

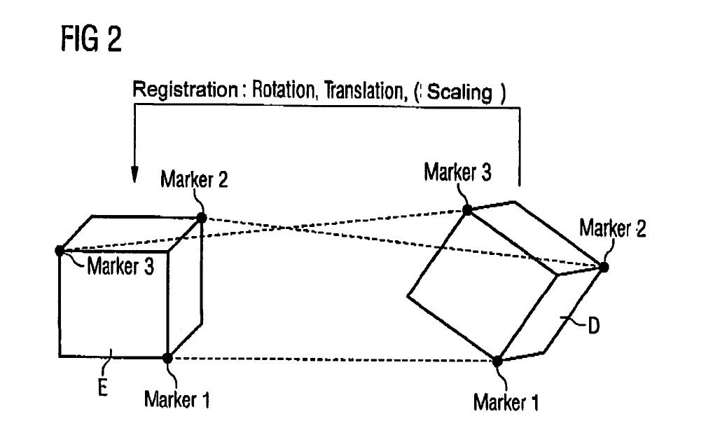

FIG. 2 explains the principle of marker-based 3D-3D registration in this case of two cube-shaped 3D images. Shown is a first cube-shaped 3D image E (for example, a preoperative 3D dataset) which has three markers (markers 1, 2, 3), as well as a second cube-shaped 30 image D (for example, an intraoperative dataset) in its original form, immediately after its creation. For registration, the marker points 1, 2 and 3 must be identified in the first 3D image E, and the corresponding points must be interactively localized by the user (for example, by clicking on the screen with the mouse) in the second 3D image D. By rotation, translation, and possibly by scaling, the coordinate transformation between the first 3D image E and the second 3D image D is determined from the corresponding point pairs (here markers 1, 2 and 3), with which the structure of both 3D images can be brought into congruence. The determination of such a coordinate transformation represents the registration.

It is not mandatory for the identification of the markers in marker-based registration ensue on the screen. Given the existence of a navigation system (navigation sensor S, see FIG. 1), and for preparation of a navigation-supported operation, a marker-based registration of a (for example preoperative) 3D image relative to the navigation system S ensues by manually tapping artificial or anatomical markers with a navigation pointer by the doctor. Since the catheter 11 is registered relative to the navigation system based on existing detectors with regard to position and location, a correlation is thus produced between catheter 11 and preoperative 3D image E. The real image of the catheter 11 thus can be calculated and visually mixed into the 3D image by the control and processing device 8. Navigation of the medical instrument in E is thus possible.

Conventional marker-based registrations have the disadvantage that often an additional operative procedure is necessary to place artificial markers. Anatomical markers are often very difficult to localize clearly, which is why a calibration with regard to a marker-based registration is often error-prone. A navigation-supported registration also has significant disadvantages: if one would want to now register, aided by navigation, intraoperatively measured 3D images with the preoperative 3D image, the marker must be manually tapped again in a navigation-supported marker-based registration at each C-arm position of the 3D image to be acquired. Such a method is in practice very error-prone and laborious. If the markers in the image are tapped in a different sequence than the anatomical markers tapped on the patient, such that it cannot be reproduced, or if the relative position of the marker has changed, false positionings ensue. Moreover, the registration must be repeated each time given a maladjustment of the navigation during the procedure.

In order to prevent or to circumvent the disadvantages cited above of a marker-based registration, the inventive method uses an image-based 3D-3D registration, at least in the registration of both 3D images (preoperative with regard to the intraoperative 3D image). It is advantageous for the registration N of the medical instrument 11 with regard to the intraoperative 3D dataset to also ensue according to the concept of a marker-less registration.

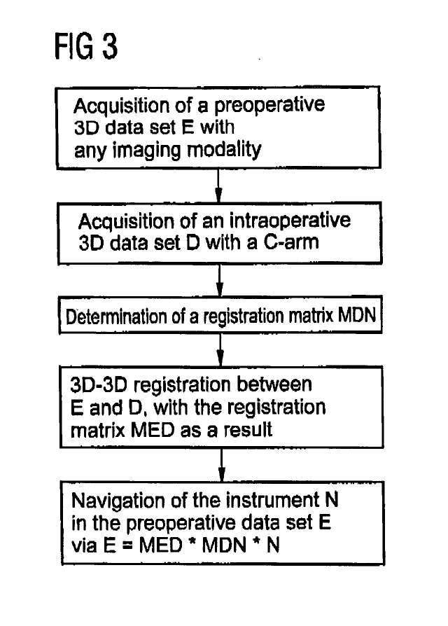

The inventive method for marker-less navigation in preoperative 3D images using an intraoperatively acquired 3D C-arm image is schematically shown in FIG. 3. The method includes five basic steps:

In a first step S1, a preoperative 3D dataset E is acquired. The 3D dataset can be acquired with any imaging modality (high-resolution or functional) (MRT, CT, PET, US, etc.). The acquisition of an intraoperative 3D image D ensues with a C-arm system in a second step S2. For such an exposure, the C-arm system is preferably operated in 3D angiography mode, so a later correlation (projection) between an individual slice of the intraoperative 3D image D and a slice of the preoperative 3D image can be determined in a simple manner. In a third step S3, a registration matrix MDN is determined by a registration N of the medical instrument 11 relative to the intraoperative 3D image. The position and location N of the medical instrument 11 in the 3D image D is specified by the matrix MDN. The determination of the matrix MDN preferably ensues according to the concept of a marker-less registration. In a fourth stop S4, a preferably image-based registration ensues between the preoperative 3D image E and the intraoperative 3D image D. A registration matrix MED is obtained as a result of this registration. The position determination (necessary for registration) of the C-arm system ensues in the intraoperative acquisition via the tool plate TP detectable by the navigation system S and affixed to the C-arm system. In a fifth step S5, the chaining of the registrations specified above ensues, making navigation of the instrument 11 in the preoperative dataset E possible according to E=MED*MDN*N.

As already mentioned, the determination of the C-arm position, and with it the position of the intraoperative 3D image, ensues by position determination of the tool plate TP by means of the navigation system S. The real tool plate position is compared with a tool plate reference position which is precisely defined relative to the navigation sensor. A non-linear C-arm deformation (typically occurring due to the inherent weight of the C-arm) is taken into account by suitable calibrations, and this can be corrected in the determination of the tool plate position given an angulation ≠0°.

In summary, the navigation of a medical instrument in a (high-resolution, functional, etc.) preoperative 3D image is achieved by the inventive method, whereby prepared results (for example the segmentation of a tumor) and/or results of a previously implemented procedure or operation plan that exist by or for the preoperative 3D image can be included in the navigation. In particular, the preoperative 3D image in turn can be the result of two superimposed preoperative 3D images (for example, anatomically- and functionally-resolved 3D images). The problems of the marker-based registration are divided by the marker-less registration.

Although modifications and changes may be suggested by those skilled in the art, it is the intention of the inventors to embody within the patent warranted hereon all changes and modifications as reasonably and properly come within the scope of their contribution to the art.

1. A method for marker-less navigation of a medical instrument comprising the steps of:
during a medical interventional procedure involving interaction of a medical instrument with a subject, intraoperatively acquiring a 3D image of the subject using a C-arm x-ray system;
bringing the medical instrument into registration with the 3D image, and thereby obtaining a first registration matrix;
bringing the 3D image into registration with respect to a pre-existing preoperative 3D image using an image-based registration, and thereby obtaining a second registration matrix; and
navigating said medical instrument in said preoperative 3D image.

2. A method as claimed in claim 1 comprising obtaining said first registration matrix by marker-less registration.

3. A method as claimed in claim 1 wherein said C-arm x-ray system has a C-arm subject to deformation, and comprising taking said deformation into account for determining said second registration matrix.

4. A method as claimed in claim 1 comprising generating said preoperative 3D image dependent on a plan for said interventional procedure.

5. An x-ray system comprising:
a C-arm on which an x-ray source and a radiation detector are mounted, said radiation detector generating electrical signals dependent on x-rays from said x-ray source incident thereon after attenuation by a subject adapted to be disposed between said x-ray source and said radiation detector; and
a control and processing unit for controlling operation of said x-ray source and for processing said electrical signals from said radiation detector, said control and processing unit, during a medical interventional procedure involving a medical instrument interacting with the subject, operating said x-ray source for acquiring an intraoperative 3D image of the subject, registering the medical instrument with respect to said intraoperative 3D image for obtaining a first registration matrix, registering the 3D image with respect a pre-existing preoperative 3D image stored in said control and processing unit, using an image-based registration, for obtaining a second registration matrix, and presenting a display of said preoperative 3D image allowing navigation of said medical instrument in said preoperative 3D image.
